(19) United States
(12) Patent Application Publication
(10) Pub. No.: US 2006/0040003 A1
(43) Pub. Date: Feb. 23, 2006

(54) Dietary supplement for supressing appetite, enhancing and extending satiety, improving glycemic control, and stimulant free

(76) Inventors: Alvin Needleman, Lancaster, PA (US); Harriet Needleman, Lancaster, PA (US)

Correspondence Address: ALVIN NEEDLEMAN, 1001 MEADOWLEAH STREET, LAS VEGAS, NV, 89145, US

(21) Appl. No.: 10/786,344

(22) Filed: Aug. 10, 2004

(51) Int. Cl. A61K 36/48; A61K 36/42

(52) U.S. Cl. 424/757; 424/776; 424/758; 424/774

(57) ABSTRACT

This invention relates to a nutritional intervention composition for enhancing satiety prior to a meal and extending satiety after a meal. The nutritional intervention composition decreases food intake producing weight loss over time. The composition consists of Niacin, Vitamin B6, Calcium, Phosphorous, Magnesium, Chromium, Chitosan, Fenugreek, Ginseng, White willow bark, Garcinia cambogia, Aloe Vera gel powder, Momordica charantia, Griffonia simplicifolia, Lagerstroemia speciosa and Vanadyl sulfate. The invention does not require stimulants or anabolic ingredients. There are three phases of activity within the composition. One, enhanced satiety through elevated serotonin. Two, improved carbohydrate metabolism, reduced blood glucose and slowed gastric emptying. Three, enhanced fiber binding of lipids and excess bile acids.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION—FIELD OF THE INVENTION

This invention relates to a method for promoting appetite suppression and enhancing satiety by elevating neurotransmitter levels, decreasing local fat absorption and stimulating glycemic control without promoting anxiety.

References Cited [Referenced By}
U.S. Patent Documents

| | | |
|---|---|---|
| 6503543 | January 2003 | Hudson |
| 4650789 | March 1987 | Pollack |
| 6436899 | August 2002 | Portman |
| 6277396 | August 2001 | Dente |
| 6025363 | February 2000 | Giles |
| 5602164 | February 1997 | Shinitzky et al. |
| 4393049 | July 1983 | Horrobin |
| 5900240 | May 1999 | Tomer et al. |

### BACKGROUND OF THE INVENTION—PRIOR ART

Sixty percent of the population of the United States is overweight. The trend toward larger portion sizes, in and out of the home, is increasing rather than decreasing. Attempts by existing weight management supplements to reverse weight gain have met with little success. Current weight management and appetite suppression supplement combinations follow repetitive historical rational and unfounded biochemical hypothesis, relying more on over aggressive marketing than fact.

Dietary supplement formulas containing a thermogenic substance or combinations of thermogenic substances. Thermogenic ingredients were chosen from a group consisting of caffeine, catechins, Ma Huang, ephedrine, synephrine, norephedrine and psuedoephedrine. The formulas often-added White willow bark, a salicylate and precursor of acetyl salicylic acid (aspirin) the rational being the synergistic (multiplier) effect of the salicylate on the thermogenic ingredients in the formula. Thermogenic products cause serious side effects including depletion of adrenal keto-steroids, anxiety, irregular heart rates, drug incompatibilities and exacerbation of high blood pressure.

A second group of dietary supplement formulas sought to reduce appetite by introducing bulk generating combinations of soluble and insoluble fibers. Formulas containing single or combinations of the dietary fiber group guar gum, pectin, acacia gum, beta-glucan, fruit fibers, vegetable fibers, legume fibers, plantago, psyllium, xanthan, agar, alginic acid, cellulose gum, methylcellulose, agarose, dextran, tragacanth, karaya, glucomannan, locust bean, konnyaku mannan and carrageenan. Unfortunately, very little weight loss or appetite control was achieved using high fiber products other than the discomfort associated with increased flatulence, gastric reflux and indigestion. A method to relieve the uncomfortable flatulence side effect is proposed by U.S. Pat. No. 5,773,427 to Charles E. Day (1996) for the inclusion of an amount of chitosan marine fiber in a specific ratio to formula fiber.

A third group of weight management products introduces natural herbal formulas, natural ingredients used alone, combined with vitamins and minerals and in formula with soluble and insoluble fibers. The most popular natural ingredient in current use is Garcinia cambogia (Hydroxycitric Acid), an exotic fruit grown in South India, often combined with chitosan, a deacytelated marine fiber and chromium picolinate. The formula rational is based on the ability of Garcinia cambogia to competitively inhibit the extramitochondrial enzyme adenosine triphosphate-citrate. As a citrate cleavage enzyme at may play an essential role in de novo lipogenesis inhibition, Garcinia cambogia is claimed to lower body weight and reduce fat mass in humans. In a 1998, study 135 subjects were randomized to either active hydroxycitric acid (G.cambogia) or a placebo for twelve weeks the results were published in JAMA 1998; 280: 1596-1600. “Garcinia cambogia failed to produce significant weight loss and fat mass loss beyond that observed with the placebo”.

Various chemical approaches have been proposed for controlling obesity. Anorectic agents, such as dextroamphetamine, are associated with undesired side effects. Indigestible materials such as mineral oil or neopentyl esters (see U.S. Pat. No. 2,962,419) have been proposed as substitutes for dietary fat. Diethylaminoethyl dextran, an ion exchange material, has been indicated to inhibit fat absorption in the body. Fischetti, Offenlegunsschrift U.S. Pat. No. 2,655,199, Chem. Abstr. 87:112005 h (1977). Garcinia acid and derivatives have been described as treating obesity by interfering with fatty acid synthesis. Swell able cross-linked vinyl pyridine resins have been described as appetite suppressants via the mechanism of providing non-nutritive bulk, U.S. Pat. No. 2,923,662. Cationic polymers such as dialkylaminoimides of alene/maleic anhydride copolymers have been described as inhibiting pancreatic lipase, U.S. Pat. No. 4,211,765.

None of the previous methods is entirely satisfactory. Controlled diet and controlled appetite remains the most prevalent technique for controlling obesity, with surgical techniques such as temporary iliary bypass surgery, being employed in extreme cases. It would be desirable to provide a new means for controlling weight gain, maintaining ideal body mass, controlling fat absorption and maintaining ideal blood glucose levels by regulating carbohydrate metabolism.

### BACKGROUND OF THE INVENTION—OBJECTS AND ADVANTAGES

Various methods have been proposed for weight control to combat obesity. One of the more common methods is the use of relatively low-fat diets containing less fat than a normal diet, although some fat is generally present even in diets considered relatively “fat Free”. Fats are solids or liquids oils generally consisting of glycerol esters with higher fatty acids. Dietary sources of fats include both animal and vegetable fats, including predominantly triglyceride esters of both saturated and unsaturated fatty acids, as well as some free fatty acids. Glyceryl tristearate, glyceryl tripalmitate and glyceryl trioleate are among the common esters.

Maintenance of fat-free or low-fat diets is difficult. The presence of fats in a great many food sources greatly limits the food sources, which can be used. Additionally, fats contribute to flavor, appearance and physical characteristics of many foodstuffs. Such factors adversely affect the acceptability of low-fat diets, and make the maintenance of such diets difficult.

This invention does not contain a thermogenic substance or a catalytic element such as white willow bark, a salicylate-containing compound, used to multiply the effect of the thermogenic ingredients to accelerate metabolism. By providing a stimulant, free supplement serious side effects are avoided as appetite is suppressed and glycolysis controlled.

This invention does not use large quantities of soluble or insoluble fiber or combinations of fibers to produce a mechanical sensation of fullness. Studies show a degree of discomfort and bloating by the user but do not show a reliable pattern of success using this approach. This invention uses an acetylated marine fiber with saponin enhanced fat absorption and slowed gastric emptying time, reducing the amount of local fat absorbed and leaving the user with a comfortable sensation of gastric satisfaction.

This invention does not use natural herbal combinations without sufficient clinical or outcome studies to provide reliable evidence of positive repeatable results. The natural products used in this invention have a history of successful performance and authenticated evidence of each ingredient's participation in the performance results of this invention. Individual ingredient clinical and biochemical validity will be identified under “claims”.

The chemical agents and controlled and dangerous drugs proposed for controlling obesity are primarily the purview of the pharmaceutical industry and therefore their distribution is confined to prescription use. Historically, chemical agents have not provided more than a 5% reduction in weight, while contributing to unwanted side effects.

This invention approaches its objectives biochemically formulating active ingredient groups, which stimulate a series of physiological events: (1) decreased local fat absorption and slowed gastric emptying time. (2) Maintenance of ideal blood glucose levels through regulated carbohydrate metabolism. (3) Enhanced and extended satiety by elevating serotonin levels. (4) Ameliorating the effects of reduced serotonin levels, improving sleep and reducing anxiety and stress. (5) Taken twice daily, immediately following meals, the invention reduces food intake by 16% to 35% in three days (results may vary between individuals).

### SUMMARY

The invention, “Dietary Supplement for Suppressing Appetite, Enhancing and Extending Satiety, Improving Glycemic Blood Levels and Stimulant Free” achieves a high level of success and consistency from the synergistic relationship between the ingredients, the action groups and the trigger mechanisms in the invention, Comprising; Aloe Freeze Dried Powder 200X, Vitamin B3, Vitamin B6, ingested proteins and carbohydrates.

### DETAILS AND DESCRIPTIONS OF THE PREFERRED EMBODIMENTS

The invention is based on three primary mechanisms of action: First, increasing serotonin levels to enhance and prolong satiety, subsequently reducing appetite and food intake by 16% to 35%. Tryptophane is a naturally occurring essential amino acid found in most protein foods, 80% of the circulating tryptophane is bound to plasma albumin with the balance of 20% circulating in the blood as free tryptophane. The invention includes a natural source of tryptophane L-5-hydroxytryptophane from the seeds of the African plant Griffonia simplicifolia, present in the range of 60 mg to 80 mg per serving, to supplement both the bound and the circulating tryptophane. To be effective tryptophane must be transported across the blood brain barrier to be metabolized into serotonin. (1) Bound tryptophane is released from plasma albumin by salicylic acid or acetyl salicylic acid (Aspirin) in a range of 0.06 Gm to 0.3 Gm. The formula ingredient “Aloe Freeze Dried Powder 200x” present in the range of 95 mg to 120 mg per serving contains between 0.009% and 0.014% salicylic acid breaking the tryptophane/plasma albumin bond and increasing the quantity of circulating free tryptophane. (2) Un-bound tryptophane, circulating tryptophane and L-5-Hydroxytryptophane are transported across the blood brain barrier when a carbohydrate source consisting of glucose, maltose, sucrose or any combination thereof is present to facilitate the transport of tryptophane and 5-L-hydoxytryptophane across the blood brain barrier. Additionally, carbohydrate intake with its insulin-releasing action helps to improve the albumin-bound tryptophane/Large electrically neutral amino acids (LNAA) ratio in favor of the tryptophane and increases the amount of tryptophane crossing the blood brain barrier into the brain. Two additional ingredients are provided in the invention to facilitate tryptophane transport into the brain and/or promote its conversion to serotonin, vitamin B3 0.15 mg to 20 mg per serving and vitamin B6 0.15 mg to 20 mg per serving. The Blood Brain barrier, in humans is a barrier that exists to allow brain functions to operate in an independent environment separate from the rest of the body to protect the sensitive nature of the Central Nervous System. For the reasons and explanations given, the invention: “The Dietary Supplement For Suppressing Appetite, Enhancing and Extending Satiety, Improving Glycemic Control and Stimulant free” is taken as three tablets with water immediately following a meal (One serving size).

Second, fiber, the absorption of local fat, reduced gastric emptying time and the relationship to satiety and decreased appetite in the invention. Chitosan is a polycationic polymer containing more than 5000 glucosamine units and is obtained by alkaline deacetylation of chitin from shellfish exo-skeletons; chitosan is present in the invention in a range of 300 mg to 600 mg per serving. Chitosan is a positively charged fiber that chemically binds to negatively charged lipids, fats and bile acids during digestion, thereby reducing fat absorption and slowing gastric emptying time. (ARS Medicina, Helsinki 1994 study showed that test subjects lost 8% of their body weight on chitosan). Dietary supplements that bind lipids aid in weight loss, with the addition of saponins to the invention, in a range of 3 mg to 5 mg per serving, (contained in the Aloe Freeze Dried Powder 200x) the capacity of chitosan to bind fat is increased. Garcinia cambogia (50% hydroxy citric acid) present in the invention in a range of 100 mg to 150 mg per serving

### ADVANTAGES OF THE PRESENT INVENTION

Accordingly, an advantage of the present invention “Dietary Supplement For Suppressing Appetite, Enhancing and Extending Satiety, Improving Glycemic Control and Stimulant Free” is that it provides a nutrition intervention composition for enhancing satiety after a meal.

Another advantage of the present invention is that it prolongs satiety for four hours after a meal.

Another advantage of the present invention is that it introduces an additional quantity of tryptophane in the form of 5-Hydroxytryptophane to support bound and free tryptophane to be metabolized into the neurotransmitter, serotonin initiating the satiety response.

Another advantage of the present invention is that it introduces an ingredient complex for lowering elevated blood glucose levels, increasing insulin in response to high carbohydrate ingestion and providing an excess of insulin to counteract Energy Homeostatic Weight Gain Bias.

Another advantage of the present invention is that it introduces a system for chemically binding lipids and bile acids, inhibiting lipogenesis and decreasing gastrointestnal transit time.

Another advantage of the present invention is that it introduces several ingredients to lower blood cholesterol.

Another advantage of the present invention is that it makes available the neurotransmitter serotonin that is metabolized to melatonin, a sleep related hormone found in the pineal gland, and results in reduced sleep latency and an improvement in the overall quality of sleep through improved sleep architecture (Boman 1988).

Another advantage of the present invention is that it makes available the neurotransmitter serotonin, which also serves well where depleted serotonin levels exist such as anxiety disorders, depression, obsessive-compulsive disorders, pain disorders and aggression.

What is claimed is:

1. A nutritional intervention composition, taken after meals (claim 4), enhancing and extending post meal satiety for reducing weight, binding with lipids and bile acids, lipogenesis inhibition, Glycemic Control and reduced sleep latency. The invention also serves well in conditions where depleted serotonin levels exist such as anxiety disorders, depression, obsessive compulsive disorders, pain disorders and aggression, comprising:
a) a source of typtophane as the modified amino acid, 5-Hydroxy tryptophane from the seed of African Griffonia simplicifolia being in the range of 50 mg to 100 mg per serving
b) a source of endogenous tryptophane naturally extraced from protein and bound to blood serum albumin and an additional 20% tryptophane circulating in the blood as free tryptophane.
c) a source of salicylic acid for releasing endogenous L-tryptophane from the serum albumen complex. In the invention Aloe, freeze-dried powder 200X being in the range of 75 mg to 125 mg per serving.
d) a source of Niacin (Nicotinic Acid) being in the range of 5 mg to 20 mg per serving.
e) a source of Vitamin B6 (pyridoxine) being in the range of 5 mg to 20 mg per serving.
f) a source of carbohydrates having a high glycemic index in an amount sufficient to facilitate the transport of tryptophane across the blood brain barrier.
I. The combinations of endogenous tryptophane (b) released from serum bound albumen (c), circulating tryptophane (b) and supplied (exogenous) tryptophane (a) comprise an amount sufficient to influence the transport of tryptophane across the blood brain barrier in the presence of carbohydrates having a high glycemic index (f). Once across the blood brain barrier (BBB), tryptophane becomes available for metabolism into serotonin, a neurotransmitter implicated in mood disorders, sleep regulation (Serotonin is in turn metabolized to melatonin, a sleep related hormone), anxiety disorders, depression, pain disorders, aggression and, principle to this invention, appetite reduction through enhancing and extending post meal satiety. Niacin (Vitamin B3/Nicotinic acid) (d) and Vitamin B6 (pyridoxine) (e) are present in the invention in amounts necessary to facilitate tryptophane uptake.

2. What is claimed is; the absorption and chemical binding of fats, lipids, bile acids, lipogenesis inhibition and the introduction of fiber for reducing weight (claim 1), comprising:
a) Chitosan, a polycationic polymer obtained by alkaline deacetylation of chitin from shellfish exoskeletons being present in the range of 450 mg to 550 mg per serving.
b) a source of saponins in the range of 3 mg to 5 mg, in the invention, from Aloe freeze dried powder 200X being in the range of 75 mg to 125 mg.
c) a source of Hydroxycitric acid (50%) from Garcinia cambogia, (an exotic fruit grown in Southern India) being in the range of 100 mg to 135 mg per serving.
II. Weight loss involves a complex series of physiological events to prevent rebound weight gain. Since the “Energy Homeostasis System” is inherently biased toward weight gain, measures are taken in this invention to circumvent a natural human tendency toward reduced metabolic rate as precipitated by reduced calorie intake. Enhanced and prolonged satiety (1) is the first step in weight loss through appetite suppression. The second step includes the absorption of fats, lipids and bile acids (1), the introduction of fiber and lipogenesis inhibition to lower body weight and reduce fat mass. Chitosan (a) is a positively charged fiber that chemically binds to negatively charged lipids, fats and bile acids during digestion, thereby reducing fat absorption, slowing gastric emptying time and interfering with glucose absorption (further contributing to satiety). In combining with bile acids, chitosan prevents the inhibition of Cholecystokinin (CCK) a peptide acting as a satiety signal in humans. The addition of Saponins (b) in Aloe freeze dried powder 200X increases the capacity of Chitosan (a) to bind with fats. Hydroxycitric acid (c) competitively inhibits the enzyme adenosine triphosphatecitrate playing an essential role in lipogenesis inhibition to lower body weight and reduce fat mass. Early trials indicated sub-par performance; however, when Hydroxycitric acid is combined with the chemical binding properties of Chitosan, lipid absorption is improved 25% to 30%.

3. What is claimed is; Glycemic control (claim 1) reducing elevated blood glucose levels in Type two Diabetes, an insulin induced advantage in the transport of free tryptophane across the Blood Brain Barrier (BBB), Carbohydrate management (1) following the ingestion of rich foods and elevated insulin levels to shift toward catabolic pathways thereby upsetting the routine bias toward weight gain, comprising:
a. Trigonella foenum graecum extract 4:1 (Fenugreek seed) being in a range of 300 mg to 400 mg per serving.
b. Momordica charantia (Bitter Melon fruit) powder being in a range of 75 mg to 125 mg per serving.
c. Lagerstroemia speciosa (Leaf) Extract being in a range of 15 mg to 3 mg per serving.
d. Chromium polynicotinate being in a range of 75 mcg to 125 mcg per serving.
e. Vanadium (Vanadyl sulfate) being in a range of 50 mcg to 100 mcg per serving.
f. Magnesium (Magnesium aspartate) being in a range of 5 mg to 15 mg per serving
III. Glycemic control (1) in the invention is multi-tiered to maintain a bias against rebound weight gain, to reduce a reversion to an anabolic pathway (3), to interfere with the secretion of the acylated peptide Grehlin, to increase insulin production enhancing the transport of tryptophane across the Blood Brain Barrier (I) and as an antidote (3) (Carbohydrate metabolizer) following the ingestion of Rich food (3). Fenugreek seed (a) shows evidence of a hypoglycemic effect due to the presence of 4-Hydoxyisoleucine a constituent making up 80% of the total content of free amino acids. Fenugreek (a) affects gastrointestinal transit, slowing glucose and directly stimulating insulin. In people with non-insulin dependent diabetes, the ingestion of fenugreek (a) can improve plasma glucose and insulin response. In people with insulin-dependent diabetes, the ingestion of Fenugreek (a) can reduce plasma glucose, glycosuria, and daily insulin requirements. Studies suggest Fenugreek (a) may decrease calcium oxalate deposition in the kidneys, and may lower serum cholesterol. Momordica charantia (b) contains an insulin-like polypeptide called polypeptide P, plant insulin, or p-insulin. P-insulin has pharmacologic effects similar to bovine insulin with an onset of action between 30 and 60 minutes, and a peak effect at about four hours. Momordica charantia (b) contains several other flavonoids with a variety of pharmacologic effects including, lowering cholesterol, raising hemoglobin and increasing white and red blood cell counts. Alpha- and Beta-momorcharin appear to have immunosuppressive activity in vitro and in animal models. A bitter melon protein from the seed and fruit called Momordica anti-human immunodeficiency virus protein (HIV) and Map30 have anti viral and anti-tumor activity in vitro. Inhibiting HIV and herpes simplex viruses', including acyclovir-resistant strains and it appears to inhibit HIV by inhibiting reverse transcriptase. MAP30 is also an N-glucosidase, which inhibits HIV ribosornal protein synthesis and might make viral and plasmid DNA into the genetic material of healthv cells. Lagerstroemia speciosa leaf (c) In 1998, a crossover, placebo-controlled clinical study was conducted at the Tokyo Jikeikai Medical School in Japan with 24 Type II diabetic human subjects. After four weeks, corosolic acid (The active ingredient in Lagerstroemia speciosa) was shown to effectively reduce blood glucose levels vs. placebo, with no adverse effects. Furthermore, even a one-time dose left a “memory-effect” for blood glucose control, lasting several days. In another study conducted at the Southwestern Institute of Biomedical Research, in Bradenton, Fla., 12 human subjects with mild Type II diabetes were studied for 22 weeks. Several forms of corosolic acid were administered to different groups, and in several dosages. It was seen that the higher the dose of corosolic acid, the greater the drop in blood glucose levels. The greatest blood glucose reduction was obtained using an oil-based soft gelatin capsule formulation of corosolic acid at a 48 mg daily dose.
In an elaborate cross-over study, 12 subjects took a placebo for two weeks, then a daily dose of 48 mg corosolic acid (two oil-based softgels of 8 mg after each meal), for 30 days. This was followed by a 45-day placebo washout period. Then the same group took 48 mg corosolic acid in a different form (two hard gelatin capsules of 8 mg corosolic acid dry powder after each meal), for 30 days. Another 45-day washout followed. The results show that corosolic acid is effective in reducing blood glucose levels, with no adverse effects.
Specifically, the average blood glucose level in the control group was 168.3 mg/deciliter.
The soft gelatin formulation of corosolic acid caused a rapid drop to an average value 115.1 mg/deciliter at the 30th day of corosolic acid (Lagerstroemia speciosa) treatment. During the washout period, the blood glucose level only slowly came back up, suggesting a memory effect of corosolic acid for up to four weeks after the termination of intake. In addition, 48 mg of corosolic acid per day continued to reduce blood glucose levels until the end of the 30-day period.
pAdditional benefits were seen, with corosolic acid restoring a normo-glycemic profile after meals. The corosolic acid group had a normal sharp decline in blood glucose levels after eating, compared to the slow decline after a meal often seen in diabetics. The diabetic symptoms of frequent thirst and urination also disappeared for those using the corosolic acid, and there was an increased ability to lose weight. This U.S. clinical study confirms the 1998 Japanese clinical study showing that corosolic acid (from Lagerstroemia Lagerstroemia speciosa) safely and effectively lowers blood glucose levels in Type II diabetics.
Multiple effect mineral ingredients in the invention contribute to formula enhancement at all three levels of activity, comprising Chromium, Magnesium and Vanadium.
Chromium polynicotinate (d) Chromium plays a role in the metabolism of glucose, and is necessary for energy production. Since this mineral assists in the production of insulin, it helps to stabilize blood sugar levels and can be beneficial both for people with hypoglycemia and diabetes. It is also critical to the synthesis of cholesterol, fats, and proteins. Chromium polynicotinate (d) is more effective than any other type of chromium, as it binds the elemental chromium to niacin (vitamin B-3) [1, d]. This provides a biologically active form of chromium, which is more absorbable in the body. Vanadium (Vanadyl sulfate) (e) is derived from the trace element vanadium. In recent studies, Vanadyl sulfate (e) has been shown to enhance many of the same anabolic processes controlled by insulin. Along with testosterone, growth hormone and thyroid hormone, insulin is a major anabolic hormone. Insulin mimicking effects by Vanadyl sulfate (e) causes glucose and amino acids to be forced into muscle to a greater degree than normal. Vanadyl sulfate (e) also helps increase glycogen storage in muscle and helps improve protein synthesis. This is the perfect anabolic environment to stimulate the metabolism of stored fat (yellow fat). Magnesium (Magnesium aspartate) (f) is the second most plentiful cation in the intracellular fluid and the most plentiful cation in the body. Up to 50% of the magnesium in the body is present in bone. Magnesium is important to the normal bone structure and it plays an essential role in more than 300 fundamental cellular reactions. Magnesium is required for the formation of cyclic AMP (cAMP) and is involved in ion movements across cell membranes. It is involved in protein synthesis and carbohydrate metabolism. E racellular magnesium is critical to both maintaining nerve and muscle electrical potentials and transmitting impulses across neuromuscular junctions. Aging and stress are thought to increase magnesium requirements. Low intake and impaired absorption of magnesium (f) have also been associated with the development of various disease states such as osteoporosis, hypertension, atherosclerotic vascular disease, cardiomyopathy, diabetes, and stroke. Symptoms of severe magnesium deficiency include convulsions, confusion, muscle weakness, abnormal muscle movements, and others. Magnesium (f) stimulates gastric motility due to the release of gastrin and Cholecystokinin (CCK) further contributing to satietv. There is evidence that magnesium is important in regulating blood pressure. Magnesium (f) deficiency has been found to cause intracellular concentrations of sodium and potassium to increase, which can lead to increased peripheral resistance and vasospasm. In cell membranes, a decreased concentration of magnesium (f) and increased calcium to magnesium ratio has also been associated with hypertension. There is also evidence that hypertensive patients with hypomagnesemia usually require more antihypertensive medications than hypertensive patients with normal magnesium levels. There is some evidence that serum magnesium (f) deficiency might play a role in both ischemic and hemorrhagic stroke. Preliminary information shows magnesium may act as a neuroprotective agent in patients diagnosed with acute stroke. Several possible mechanisms of neuroprotection exist, including noncompetitive N-methyl-D-aspartate antagonism and calcium channel antagonism. In patients with congestive heart failure, there is evidence magnesium reduces coronary vascular resistance, increases coronary artery blood flow, has antiarrhythmic effects, and improves cardiac indexes. There is evidence that low magnesium levels play a role in diabetes and migraine headaches. Magnesium blood levels play a role in insulin resistance. There is also evidence that low dietary intake of magnesium (f) increases the risk of developing type 2 diabetes. Effects of magnesium on serum lipids may be due to decreased lipolysis and increased lipoprotein lipase activity. Intracellular levels of magnesium, measured in erythrocytes and leukocytes, have been found to be lower in women with premenstrual syndrome (PMS), leading to the use of magnesium supplements for PMS. Magnesium (f) is reported to be an antagonist at N-methyl-D-aspartate (NMDA) receptors, which are involved in the potentiation of pain. This effect and magnesium's depressant effects on nerves and smooth muscle are thought to contribute to the possible effects of magnesium (f) in relieving symptoms associated with migraines, postoperative pain, neuropathic pain, erythromelalgia, Raynaud's Phenomenon, and other vascular disorders and pain syndromes. There is some evidence that magnesium metabolism is a factor in renal stone formation and prevention.

4. What is claimed in the invention is, a unique dosage schedule: the invention may be taken before or after meals to enhance and prolong satiety. Adult dosage three tablets twice daily.

5. What is claimed in the invention is; the invention may be taken (orally) after the ingestion of calorie rich food (Hgh Carbohydrate) as an antidote to interfere with calorie intake in the interest of weight management.

6. What is claimed in the invention is the influence of high carbohydrate on transport and conversion of tryptophane to serotonin promoting satiety (claim 5).
